(19) United States
(12) Patent Application Publication
(10) Pub. No.: US 2024/0287435 A1
(43) Pub. Date: Aug. 29, 2024

(54) METHOD AND DEVICE FOR MONITORING THE CONTENTS OF MIXED REACTORS

(71) Applicant: AQUILA BIOLABS GMBH, Baesweiler (DE)

(72) Inventors: Konrad Herzog, Herzogenrath (DE); Julian Sidram, Baesweiler (DE); Felix Kortmann, Baesweiler (DE)

(21) Appl. No.: 18/573,974

(22) PCT Filed: May 12, 2023
(86) PCT No.: PCT/EP2023/062790

§ 371 Date: Dec. 22, 2023

Related U.S. Application Data
(60) Provisional application No. 63/433,229, filed on Dec. 16, 2022.

(30) Foreign Application Priority Data
Jun. 13, 2022 (DE) 10 2022 002 116.1
Apr. 28, 2023 (DE) 10 2023 111 143.4

(51) Int. Cl. C12M 1/34; C12M 1/06

(52) U.S. Cl. CPC C12M 41/30; C12M 27/02; C12M 41/26

(57) ABSTRACT

Methods and devices for monitoring the content of mixed reactors, where the reactor has at least one property to be monitored that influences at least one signal of at least one sensory component, where the at least one sensory component moves within the reactor so that it is not permanently located in the detection region of the at least one measuring arrangement, and detection is carried out by at least one measuring arrangement while the at least one sensor is located in the detection region.

### TECHNICAL FIELD

The invention relates to a method and a device for monitoring the content of mixed reactors. In particular, it is applicable for monitoring the content of mixed reactors with high demands on sterility and purity, as well as in applications with reactors of complex geometry or small size and associated limited accessibility for measuring devices. Applications of the invention can thus be found, for example, in the process monitoring of cell cultures or chemical reactions, of work-up, filtration and formulation processes of pharmaceutical, biological or chemical products, and in the monitoring of storage processes.

### BACKGROUND

Mixed reactors are used in many areas of chemical, biological and biotechnological industry and research, in particular for carrying out chemical or biochemical reactions and syntheses, for cultivating living cells, for work-up, filtration and formulation of pharmaceutical, biological or chemical products, and for storing a wide variety of constituents of such processes. The content of mixed reactors is monitored in particular for the purposes of research, process development and optimization, process control and regulation as well as characterization and quality assurance, wherein various properties of the reactor content or their correlates are detected by means of suitable sensors and measuring arrangements and are often recorded over the duration of the process.

In many cases, the detection of certain properties of the reactor content requires the use of multi-stage measuring chains since the properties to be monitored are not available directly as an electronically detectable signal, for example as an electromagnetic wave, electrical voltage or electrical current. Rather, sensors are used which contain at least one sensory component which is a constituent of at least one sensor which is located inside the reactor and which reacts to the property of the reactor content to be monitored and, in dependence thereon and possibly via intermediate steps of further sensory components, generates a signal which is detectable by a suitable measuring arrangement. A variety of approaches are known to the person skilled in the art for this purpose, in particular, but not exclusively, the sensory use of the dependence of optically detectable properties (e.g. absorption and emission spectra, luminescence lifetimes, quantum yields, polarization behavior, plasmon resonance) of suitable dyes on the properties of the reactor content (e.g. pH, temperature, oxygen saturation, substrate, metabolite and product concentrations). The connection of such dyes or other metrologically detectable sensory components with sensory components (e.g. enzymes, aptamers, antibodies) which are not directly detectable by a suitable measuring arrangement but, for example, influence a metrologically detectable sensory component via by-products, co-substrates (e.g. protons as pH, oxygen), dissociation and association reactions or conformational changes, are also known to the person skilled in the art.

The combination of suitable sensors and measuring arrangements therefore principally enables the monitoring of the content of mixed reactors, with the properties to be monitored being converted by suitable sensors inside the reactor into a signal that can be detected electronically by the measuring arrangement. If the reactor content consists of several phases (e.g. liquid or liquid-like reaction mixture and overlying gas phase in the headspace of the reactor), the monitoring of the properties of individual phases is often important in the application (e.g. oxygen concentration in the reaction mixture, oxygen concentration in the gas phase of the headspace).

### PRIOR ART

Methods and devices are known from the prior art which enable the monitoring of the content of mixed reactors by comprising at least one sensory component which is a constituent of at least one sensor located inside the reactor and reacts to the property of the reactor content to be monitored and, in dependence thereon, generates a signal which is detectable by a suitable measuring arrangement.

An overview of the prior art for monitoring oxygen concentration in reactor contents is provided in the review article by Wang Xu-dong and Otto S. Wolfbeis (“Optical Methods for Sensing and Imaging Oxygen: Materials, Spectroscopies and Applications”. Chem. Soc. Rev. 43, no. 10 (2014): 3666-3761. https://doi.org/10.1039/C4CS00039K), with Chapter 11 reflecting the prior art on sensor formats.

A comparable overview for monitoring pH in reactor contents is provided in the review article by Andreas Steinegger, Otto S. Wolfbeis, and Sergey M. Borisov (“Optical Sensing and Imaging of PH Values: Spectroscopies, Materials, and Applications.” Chemical Reviews 120, no. (Nov. 25, 2020): 12357-489. 22 https://doi.org/10.1021/acs.chemrev. 0c00451), with Chapter 14 reflecting the prior art on sensor formats.

Further comparable or derived methods and devices for monitoring other properties of the reactor content are known to the person skilled in the art from the technical literature.

The above-mentioned review articles describe the use of optically detectable sensory components, wherein at least one of their optical properties changes depending on the property of the reactor content to be monitored (for example, pH or oxygen concentration). The optical detection is performed via suitable measuring arrangements which typically have at least one light source and at least one light detector. The two review articles disclose different sensor formats and associated concepts and embodiments of the optical measuring arrangements. Layer-like sensor formats are disclosed which are attached onto the inside of the reactor wall, onto the inside of optical windows of immersion probes, or onto optical fibers and other totally reflective light guides in contact with the reactor content. The associated measuring arrangement is in optical contact with the sensors and the sensory components contained therein. Furthermore, microparticulate and nanoparticulate sensor formats are disclosed. In some embodiments, these particles are embedded in polymers and, comparable to the aforementioned layer-like sensors, are attached onto the inside of the reactor in a stationary manner, with the associated optical measuring arrangement also being stationary. In other embodiments, these particles are added to the reactor content and either concentrated locally in the optical field of view of the measuring arrangement by magnetic interactions, or else added in such a high concentration that such magnetic concentration is not necessary to achieve sufficiently strong optical signals.

The article by David Flitsch, Tobias Ladner, Mihaly Lukacs, and Jochen Buchs (“Easy to Use and Reliable Technique for Online Dissolved Oxygen Tension Measurement in Shake Flasks Using Infrared Fluorescent Oxygen-Sensitive Nanoparticles.” Microbial Cell Factories 15, no. 1 (December 2016): 45. https://doi.org/10.1186/s12934-016-0444-4) discloses a combined embodiment for monitoring the oxygen concentration in the reactor content, with homogeneously distributed sensor particles in the reaction mixture and a sensor stationarily attached to the inside of the reactor wall. The optical properties of both sensor formats are detected via measuring arrangements with a stationary fiber outside the reactor. The article shows a serious disadvantage of sensors stationarily attached in the reactor in applications where the sensor is not permanently in contact with the reaction mixture. In such a case, due to its inherent inertia, the sensor reacts to properties of the reaction mixture as well as to properties of the gas phase in the headspace of the reactor, resulting in a mixed signal that deviates from the actual signal of the respective phase.

A wide variety of methods and devices for monitoring the content of mixed reactors is also known from the patent literature. They are all similar with regard to the stationary attachment of sensors on the inside of the reactor wall or other constituents of the reactor, with each sensor being assigned a suitable measuring arrangement in a stationary manner, either as a complete measuring arrangement or else via a component of the measuring arrangement which is stationary with respect to the respective sensor (for example in the form of an optical fiber).

DE10101576A1 discloses an optical sensor for determining at least one parameter in a sample, which sensor contains an indicator material of short decay time responsive to the parameter and a reference material of long decay time non-responsive to the parameter and which serves for detecting a measurement signal indicative of the parameter to be determined on the basis of the jointly detected luminescence responses of the indicator and reference materials. The indicator and reference materials are immobilized on a common support and in contact with the reactor content. The carrier can be a film, a cassette through which the sample is to flow, or a planar or fibrous light guide. In the case of a plurality of sensors, the sensors can be arranged next to each other and separated from each other on the carrier or they can also be mixed.

EP1397672A1 discloses a device for detecting oxygen comprising a support having a plurality of wells for receiving samples, wherein stationary oxygen sensors are provided on the inner sides of the wells, comprising particles containing a luminescent dye quenchable by oxygen and a gas-permeable and substantially water-impermeable first matrix, and a substantially water-permeable second matrix, wherein the particles are dispersed in the second matrix. The device is configured as a microtiter plate and a culture plate for culturing cells.

EP3821230A1, DE102018105174A1, EP2321052A1 and U.S. Pat. No. 20,081,71383A1 disclose devices and methods for monitoring the content for different types of reactors (channels, bags, stirred vessels), wherein the sensors are stationarily attached onto a sensor carrier and this sensor carrier is fastened in the reactor wall in such a manner that the sensor comes into contact with the reactor content. The optical properties to be detected of the sensors stationarily installed in the reactor wall are detected via suitable stationary optical measuring arrangements or via optical fibers mounted in a correspondingly stationary manner as components of suitable optical measuring arrangements.

The methods and devices for monitoring the content of mixed reactors known from the prior art disclose sensors which, via suitable sensory components, convert at least one property of the reactor content to be monitored into a signal which can be detected electronically by the measuring arrangement. The vast majority of these methods and devices use sensors stationarily connected to the inside of the reactor wall or reactor internals in combination with likewise stationary measuring arrangements inside or outside the reactor for detecting the sensor signal.

The concept of stationary sensors and measuring arrangements is accompanied by some serious disadvantages. For example, the parallel monitoring of a plurality of properties of the reactor content via a plurality of suitable sensors also requires a parallelization or duplication of the associated measuring arrangements. The resulting increase in space requirements for the measuring arrangements limits the miniaturization of reactors, which is often of interest for screening and process development work and limits the number of reactor content properties that can be monitored in parallel in miniaturized reactors. Furthermore, the attachment of stationary sensors onto internal reactor parts or walls, in particular under sterile conditions, is very costly. Even for the simplest reactors such as shake flasks, no robust and user-friendly systems for attaching stationary sensors onto the inside of the reactor walls exist to date. Non-sterile attachment of stationary sensors onto internal reactor parts or walls in combination with subsequent sterilization is also disadvantageous since many common sterilization processes adversely affect the behavior of sensory components.

Another disadvantage of stationary sensors in the case of measuring arrangements located outside the reactor is the required accuracy in positioning and orientation between the inside sensor and the outside measuring arrangement, which can often only be achieved by complicated positioning aids. In addition, stationary sensors in mixed reactors are in some cases exposed to high shear forces, which can have a life-limiting effect or even decompose the sensors thereby contaminating the reaction mixture.

Some disadvantages of stationary sensors can be avoided by using micro- and nanoparticles in the reaction mixture as known from the prior art. These particles are either freely and uniformly distributed in the reactor content or reaction mixture, respectively, or they are magnetically focused within the detection region of the measuring arrangement for signal detection and therefore appear in both cases to the measuring arrangement as a stationary sensor. However, compared to the sensor stationarily attached onto the inside of the reactor wall, the above-described erroneous detection of mixed signals of the properties to be monitored from different phases of the reactor content does not occur due the clear phase assignment of the sensor particles. Furthermore, the homogeneous distribution of the sensor particles in the reactor content or reaction mixture, respectively, and the magnetic focusing of the sensor particles in front of the measuring arrangement simplify both the insertion of the sensors into the reactor content and the positioning and orientation of the measuring arrangement.

Nevertheless, the use of freely distributed sensor particles also has considerable disadvantages. For example, with increasing reactor volume, increasing sensor particle quantities are also required to achieve the sensor particle concentrations in the reactor content necessary for correct signal detection by suitable measuring arrangements, so that monitoring costs increase significantly for larger reactor volumes. In addition, the use of homogeneously distributed as well as magnetically focused sensor particles makes it difficult to monitor a plurality of properties of the reactor content in parallel via a plurality of suitable sensor particles. In many cases, the reason for this is the mutual interaction or influence of the sensor signals (for example, due to filter or scattering effects in the case of optical sensors with absorption, luminescence or scattered light detection) of different sensor particles in the same reaction mixture. In addition, the use of identical sensory components detectable by suitable measuring arrangements is prevented in sensors with multistage measuring chains. For example, the use of sensors with the same oxygen-quenchable luminophore but different oxygen-consuming enzymes for parallel monitoring of, for example, glucose, lactate and glutamine with the particle sensors known from the prior art is not possible, since no currently available measuring arrangement could distinguish by which of the different substances as properties of the reactor content to be monitored the detected signal of the luminophore is influenced. This disadvantage does not exist with stationarily attached sensors since the combination of stationary sensor and stationarily assigned measuring arrangement implicitly also assigns the properties monitored in parallel and keeps them distinguishable.

DE102004017039A1 discloses a method and a device for detecting process parameters of reaction liquids in a plurality of microreactors, which are continuously shaken at least until the completion of the reaction in all microreactors. The process parameters in the microreactors are detected during the reaction using at least one sensor optics constituent of the measuring arrangement. In some embodiments, the sensor optics is movable via a positioning unit. Also disclosed are embodiments in which the reaction liquids in the microreactors include at least one chemical sensor material, and the chemical sensor material is attached to at least one inner surface of the microreactor. It is further disclosed that each sensor optics is not moved, at least during detection of the process parameters, so that the shaken microreactors move relative to each sensor optics. Furthermore, embodiments are disclosed in which the radiation source of the sensor optics is a flash lamp whose pulse frequency is adapted to the shaking movement such that the light flashes strike the microreactor always at the same location during the shaking movement. Also disclosed is an embodiment in which a high flash and detection frequency, which is higher than the shaking frequency of the shaker, is used to record many measurement points to prevent beating of the measurement signal. The collectively disclosed combination of movable sensor optics as a component of a measuring arrangement, stationary chemical sensors mounted on the inner wall of the reactor and a pulsed radiation source of the sensor optics, synchronized with the shaking movement, basically allows the detection of a plurality of stationary sensors, also by using identical optical sensory components and at the same time miniaturizing the reactor. Nevertheless, the disadvantages of the time-consuming attachment of stationary sensors onto internal reactor parts or walls under sterile conditions and the disadvantages of sterilization after attachment still remain. An additional disadvantage is the need to synchronize the radiation source to the shaking movement, which results from the relative movement between the shaken stationary sensor and the unshaken measuring arrangement. Disadvantageously, in the case of the use of sensors attached stationarily in the reactor, there is also the danger of the above-described detection of mixed signals from different phases of the reactor content. In the case of the use of sensor particles freely distributed in the reaction mixture, the disadvantages described above arise with regard to the parallel detection of different properties of the reactor content via the use of a plurality of, or else partly identical, sensory components.

All methods and devices disclosed in the prior art for monitoring the content of mixed reactors have at least one of the above-described disadvantages with regard to scalability of the reactor volume, detection of a plurality of properties of the reactor content or complexity in handling and compliance with sterility and purity requirements of the processes to be monitored.

### OBJECT OF THE INVENTION

It is therefore an object of the present invention to provide a method by means of which the monitoring of the content of mixed reactors with sensors inside the reactor can be carried out robustly and simply in handling while simultaneously providing good scalability into large and small reactor volumes and the possibility of detecting a plurality of properties of the reactor contents.

### BRIEF SUMMARY

In one aspect of the invention, a method for monitoring the content of mixed reactors is provided, wherein the reactor content has at least one property to be monitored, wherein the at least one property to be monitored directly or indirectly influences at least one signal of at least one sensory component, wherein the at least one signal of the at least one sensory component is detectable by at least one measuring arrangement, and wherein the at least one sensory component is a constituent of at least one sensor. Th method is characterized in that the at least one sensor is not stationary and performs a movement in the reactor, and that at least one detection of the at least one signal of the at least one sensory component is carried out by at least one measuring arrangement while the at least one sensor is located in the detection region of the at least one measuring arrangement, and that the at least one sensor is not permanently located in the detection region of the at least one measuring arrangement.

In some embodiments, the detection time of the at least one signal is shorter than the dwell time of the at least one sensor in the detection region of the at least one measuring arrangement.

In some embodiments, the monitoring is carried out using at least one converting component contained in the sensor, wherein the at least one converting component directly or indirectly influences at least one sensory component contained in the sensor and the signal thereof detectable by at least one measuring arrangement.

In some embodiments a plurality of different sensors is used to monitor a plurality of different properties of the reactor contents.

In some embodiments, at least one marker signal of at least one marker contained in the sensor is detected by at least one measuring arrangement.

In some embodiments, a measuring arrangement includes a plurality of signal detectors and/or a plurality of signal exciters detects a plurality of signals 8 and/or a plurality of marker signals.

In some embodiments, the addition of at least one sensor into the reactor takes place before the start of the process to be carried out in the reactor.

In another aspect of the invention, a device for carrying out the methods is provided, the device having a reactor with reactor content, at least one non-stationary sensor, and at least one measuring arrangement for detecting at least one signal of at least one sensory component contained in the sensor, and the volume of at least one detection region of at least one measuring arrangement, which volume overlaps with the reactor content, is smaller than the volume of the reactor content.

In some embodiments, the volume of at least one detection region of at least one measuring arrangement, which volume overlaps with the reactor content, is smaller than at least one of the sensors detectable by the measuring arrangement.

In some embodiments, the device includes a plurality of non-stationary sensors.

### BRIEF DESCRIPTION OF THE DRAWINGS

FIG. 1 shows a schematic illustration of the method according to the invention using two sensors 5 with different sensory components 7.

FIG. 2 shows a schematic illustration of a sensor 5 according to the invention and a device according to the invention for carrying out the method according to the invention using the example of a T-flask as reactor 1 for cultivating cells.

FIG. 3 shows a schematic illustration of a sensor 5 according to the invention for carrying out the method according to the invention using the example of a shake flask as reactor 1 for cultivating cells with addition system 18 for sensors 5.

FIG. 4 shows a schematic illustration for carrying out the method according to the invention on a shake flask as reactor 1 for cultivating cells using the sensor 5 according to the invention shown in FIG. 5.

FIG. 5 shows a schematic illustration of two sensors 5 according to the invention and a device according to the invention for carrying out the method according to the invention on a stirred tank reactor as reactor 1.

FIG. 6 is a schematic representation of a sensor 5 according to the invention in core-shell design with adapted geometry for use in orbitally shaken systems.

### DETAILED DESCRIPTION

### Definitions

To ensure clarity of some terms used in the description, they are defined and explained below and in the course of the description.

A reactor is a container which can be used in particular for the cultivation of organisms or for carrying out chemical and biochemical reaction processes. Further fields of application of reactors include, amongst others, biocatalytic processes using organisms and/or biomolecules, as well as other chemical and/or physical processes, wherein the term process comprises all types of conversion, separation, combination, mixing, size change and storage of, in particular, chemical substances, organisms, particles, solutions, emulsions and foams. Reactors within the meaning of the invention comprise, in particular, stirred tank fermenters and reactors, bubble column fermenters, shake flasks, T-flasks, microtiter plates, deep well plates, shake barrels, fermentation bags, multipurpose tubes, and cell culture dishes. Reactors can be closed or open with respect to their environment.

The reactor content comprises all matter that is inside the outer shell of the reactor. In the case of a reactor that is open with respect to the environment, the reactor content is delimited by the inner wall of the reactor as well as by the hypothetical closure surface which represents the transition between the reactor interior and the environment. The reactor content is composed of one or more phases, which are in particular formed as fluids (gases, liquids) or phase mixtures with fluidic character (foams, emulsions, suspensions, powder spills). In many fields of application of the invention, the reactor content is formed by two phases, namely by the reaction mixture in which the major part of the process to be carried out takes place, and by the overlying headspace, which is usually formed as a gas phase. According to the invention, each phase of the reactor content can be a pure substance or a mixture of substances. The reactor content, and thus in particular also the reaction mixture and the headspace, have physical, chemical, biological or other properties which can be the target object of monitoring. Within the meaning of the invention, sensor-based monitoring of one or more properties of the reactor content, the reaction mixture, the headspace or their constituents can be performed by detecting and determining the property itself or by detecting correlates of the property. In this context, correlates within the meaning of the invention are any phenomena, processes, signals, properties or environmental conditions of suitable sensors, sensory components or converting components that correlate with the property to be monitored. Properties within the meaning of the invention are qualitative or quantitative quantities suitable to describe the state of a thing, in particular but not exclusively, substance and particle concentrations, environmental parameters such as temperature and pressure, optical parameters such as luminescence lifetime, emission, absorption or scattering intensity and wavelengths, other physical parameters such as emissivity, impedance, electrical capacitance, inductance and conductivity, biological parameters such as expression rates, metabolic pathway activities, division rates or viability, and many more. Within the meaning of the invention, monitoring of properties of the reactor content or its constituents can also be performed via single or joint detection of a plurality of correlates and their combination with suitable mathematical calculation and evaluation methods or with other algorithms.

Mixing of the reactor content refers to any method of influencing the reactor content in such a manner that at least two states of the distribution of the reactor content and its constituents in the reactor, recorded at different times, do not resemble each other. Common mixing processes use in particular mechanical, thermal or thermodynamic processes for this purpose, in particular but not exclusively, shaking methods, stirring methods, pumping methods and diffusion methods.

Sensors within the meaning of the invention are devices which are suitable for converting at least one property of the reactor content or at least one of its constituents into at least one signal which can be detected, if necessary by means of excitation, by suitable measuring arrangements. For this purpose, sensors within the meaning of the invention comprise at least one sensory component which has at least one property or a corresponding correlate which can be detected by the measuring arrangement and is influenced by the property to be monitored of the reactor content or at least one of its constituents. The influence by the at least one property to be monitored can take place directly or indirectly, in particular interaction chains with different converting components or with other properties or constituents of the reactor content. Within the meaning of the invention, the detection of properties or correlates by measuring arrangements takes place via at least one signal of at least one sensory component, which in some cases must be excited by the measuring arrangement. Sensory components within the meaning of the invention are in particular, but not exclusively, dyes, fluorophores, luminophores, nano- or microparticles of metals or semiconductors and other structures that can generate or influence optical or other electromagnetic signals, as well as combinations of the aforementioned or similar components with each other and systems that contain at least one of the aforementioned components.

Sensors within the meaning of the invention can further contain one or more converting components. A converting component within the meaning of the invention has at least one property ora corresponding correlate, which is or are influenced directly or indirectly (via another converting component) by the property to be monitored of the reactor content or at least one of its constituents and which, however, is not detectable by a measuring arrangement, but can directly or indirectly (via another converting component) influence at least one property and thus at least one signal of at least one sensory component located in the vicinity of the converting component. Converting components within the meaning of the invention can be linked via their interactions with each other or with sensory components to form interaction chains of any length in order to convert changes in at least one property of the reactor content or at least one of its constituents into a signal that can be detected by at least one measuring arrangement. Converting components within the meaning of the invention advantageously have a high specificity with respect to the property to be monitored or the interacting other converting components or sensory components and are used to monitor in particular substance concentrations. Converting components within the meaning of the invention are in particular enzymes, catalysts, nucleic acids, aptamers, ribozymes, antibodies and other selectively binding proteins or structures, as well as combinations of the aforementioned or similar components with each other and systems containing at least one of the aforementioned components. Within the meaning of the invention, the sensory component influenced by at least one converting component is preferably located in spatial proximity to the converting component influencing it, in particular to enable effective interaction between the two, for example by local changes in substance concentrations (in particular oxygen or proton concentration) or by energy, electron or proton transfer processes (in particular quenching, FRET, PET, electroluminescence, reactions).

Sensors within the meaning of the invention can further include markers which generate at least one marker signal so that in particular the location, position and identity of the respective sensor can be determined by suitable measuring arrangements via the detection of the at least one marker signal.

Sensors within the meaning of the invention can have at least one sensor matrix that provides mechanical stability as a structural component, serves to maintain the sensor-internal arrangement of sensory components, converting components, and markers, can embed them in the sensor, can separate them from one another, and can thus segment the sensor. A sensor matrix can also be used to give the sensor a specific shape and maintain it. Moreover, sensor matrices within the meaning of the invention can be used to adjust selective mass transfer, for example, through defined pore sizes, polarity or surface charge. Sensor matrices within the meaning of the invention are considered to be in particular, but not exclusively, all types of polymers, hydrogels, membranes, as well as amorphous, semi-crystalline or crystalline solids. Several identical or different sensor matrices can be used in one sensor.

A measuring arrangement within the meaning of the invention is any device or combination of devices suitable for detecting at least one signal of at least one sensory component or at least one marker signal of at least one marker. In this respect, any property or its correlate of a sensory component or a marker that can be detected by a measuring arrangement is a signal or marker signal, respectively, within the meaning of the invention. For the purpose of detecting signals and marker signals, a measuring arrangement comprises at least one signal detector. If the signal or marker signal to be detected must be excited, a suitable measuring arrangement further comprises at least one signal exciter. Signals to be excited within the meaning of the invention are in particular, but not exclusively, optical signals such as luminescence, absorption and scattering intensities, degrees of luminescence polarization, luminescence lifetimes and impedance signals such as capacitance, inductance or conductivity. Signals can be detected in a frequency-dependent or time-dependent manner. Signals can be detected by modulating the signal excitation. Derived signals (e.g. spectra, ratiometric signals, decay curves, Bode plots, time series) can be formed from individual detected signals (for example, intensities). In order to form derived signals, suitable electrical circuits or computers with suitable software and algorithms can be used which, within the meaning of the invention, are constituents of the corresponding measuring arrangement. Any electronic device which can store data (in particular arithmetic and logic data) and process them on the basis of programmable rules counts as a computer. Computers within the meaning of the invention are in particular, but not exclusively, microcontrollers, microprocessors, system-on-a-chip computers (SoC), PCs and servers.

Signals which can be detected by suitable measuring arrangements are detected in the detection region of the measuring arrangement, wherein the detection region results from the detection region of at least one signal detector and, in the case of a required signal excitation, the excitation range of at least one signal exciter. Within the meaning of the invention, a measuring arrangement detects all signals that can be detected by it within its detection range, i.e., not only the signals of sensory components or the marker signals of markers, but also background and ambient signals which can be eliminated by suitable methods if required. In this respect, measuring arrangements within the meaning of the invention also detect signals, in particular ambient and background signals, when there is no sensor located within their detection range.

The dwell time within the meaning of the invention is the time during which a particular sensory component of a sensor is located within the detection region of a measuring arrangement that is capable of detecting a signal of the sensory component.

The detection time within the meaning of the invention is the time required by a measuring arrangement to detect a particular signal exactly once.

A signal detector within the meaning of the invention is any device or combination of devices suitable for detecting and digitizing at least one signal of at least one sensory component or at least one marker signal of at least one marker. Signal detectors within the meaning of the invention therefore comprise, in particular but not exclusively, electrodes, antennas, photodiodes, phototransistors, photoresistors, CCD and CMOS arrays, optical elements such as filters, gratings, lenses, fibers and apertures, but also amplifier and signal shaping circuits, analog-to-digital converters, computers, and combinations of all the aforementioned elements.

A signal exciter within the meaning of the invention is any device or combination of devices suitable for exciting at least one signal of at least one sensory component or at least one marker signal of at least one marker. Signal exciters within the meaning of the invention therefore comprise, in particular but not exclusively, electrodes, antennas, LEDs, flash lamps, lasers, optical elements such as filters, gratings, lenses, fibers and apertures, but also driver and modulation circuits, digital-to-analog converters, computers, and combinations of all of the aforementioned elements.

Within the meaning of the invention, the addition of at least one sensor according to the invention into a reactor is any systematic transfer of at least one sensor from outside the reactor into the reactor interior, in particular into the reactor content or at least one of its constituents, such as, for example, into a reaction mixture or the headspace.

A storage medium within the meaning of the invention is any substance or mixture of substances with which at least one sensor according to the invention is systematically brought into contact or kept in contact during its storage or otherwise safe-keeping prior to its addition into a reactor, in order to systematically influence or maintain certain properties or behaviors of the at least one sensor. Storage media according to the invention are in particular, but not exclusively, air, defined gas mixtures, inert gases, aqueous solutions, buffer solutions, salt solutions, mixtures of organic and aqueous constituents, emulsions, foams and also powders.

Fins within the meaning of the invention can be constituents of sensors according to the invention. A fin within the meaning of the invention is any device suitable for systematically influencing the movement behavior of sensors according to the invention within the reactor content or at least one of its constituents or else the flow behavior of fluids in the reactor interior along sensors according to the invention. Thus, fins within the meaning of the invention are considered to be in particular but not exclusively fluidic structural elements, such as fins, notches, gaps, grooves and perforations, but also buoyancy elements such as weights, cavities, foams or otherwise closed-porous structures.

An agitator within the meaning of the invention is any device in or on the reactor which can either itself be set in motion, or which sets the reactor in motion, in order to cause a mixing movement, in particular as a convective mass transport in the reactor content or in at least one of its constituents, for example in the reaction mixture or in the headspace.

### Solution

According to the invention, the solution of the object for monitoring the content of mixed reactors, wherein the reactor content has at least one property to be monitored and the at least one property to be monitored influences at least one signal of at least one sensory component and the at least one signal of the at least one sensory component is detected by at least one measuring arrangement, is achieved by a method in which at least one sensor containing the at least one sensory component is not stationary and moves within the reactor so that the at least one sensor is not permanently located in the detection region of the at least one measuring arrangement, and at least one detection of the at least one signal of the at least one sensory component is carried out by the at least one measuring arrangement while the at least one sensor is located in the detection region of the at least one measuring arrangement.

According to the invention, at least one signal of the at least one sensory component detected while the at least one sensor was in the detection region of the at least one measuring arrangement is used for monitoring or determining the at least one property of the reactor content to be monitored. According to the invention, this monitoring can also be carried out via the detection of correlates of the property to be monitored.

In contrast to the micro- and nanoparticle sensors known from the prior art, the sensors according to the invention are not uniformly distributed in the reactor content or reaction mixture so that the sequential or parallel detection of a plurality of sensors with different sensory components is made possible by the same or by a plurality of suitable measuring arrangements, without the sensors according to the invention influencing or interfering with each other with respect to their detection by suitable measuring arrangements. Moreover, the method according to the invention of non-uniformly distributed sensors moving within the reactor enables a more cost-effective monitoring of larger reactors since there is no need to add larger quantities of sensors to the reactor content to achieve detectable measurement signals, as is necessary for micro- and nanoparticle sensors.

The use according to the invention of non-uniformly distributed sensors moving within the reactor advantageously enables sequential detection of the signals from the sensory components located in each case in the sensors so that, in contrast to the stationary sensors known from the prior art, the detection of different signals from different sensors and sensory components for monitoring different properties of the reactor content is possible with a reduced number of measuring arrangements, and such monitoring can also be carried out on miniaturized reactors.

In contrast to stationary sensors, the method according to the invention also simplifies the use of sensors in the reactor since the sensors according to the invention can simply be added during the filling of the reactor and do not have to be attached beforehand onto the inner walls or other reactor internals as in the case of stationary sensors.

According to the invention, the volume of at least one detection region of at least one measuring arrangement, which volume overlaps with the reactor content, is smaller than the volume of the reactor content. In an advantageous configuration of the invention, the volume of each detection region of each individual measuring arrangement, which volume overlaps with the reactor content, is smaller than the volume of the reactor content and, in particular, is so small that for each sensor that is used and is detectable by the respective measuring arrangement, the one state of the method according to the invention can be achieved in which the respective sensor is not located in the detection region of the respective measuring arrangement. In a further advantageous configuration of the invention, the detection region of each individual measuring arrangement is smaller than or equal to the volume of the reactor content or reaction mixture minus the volume of all sensors which can be detected by the respective measuring arrangement and which are currently located in the reactor or reaction mixture and could get inside the detection region of the respective measuring arrangement by movement.

According to the invention, the detection region of each individual measuring arrangement is at least large enough that the smallest sensor to be detected by the respective measuring arrangement can still be detected by it.

In some embodiments of the invention, at least one detection region of at least one measuring arrangement is smaller than at least one of the sensors detectable by the measuring arrangement. In some embodiments of the invention, each detection region of each measuring arrangement is smaller than the smallest sensor detectable in the corresponding detection region. Furthermore, in some embodiments of the invention, the detection region of each than the smallest contiguous measuring arrangement is sensor region within a sensor that is detectable in the corresponding detection region and contains the same type of sensory components.

In an advantageous configuration of the invention, the at least one signal or a plurality of signals or the totality of the signals of a sensory component that can be detected by at least one suitable measuring arrangement can be detected even if the sensor does not completely fill the detection region of the at least one measuring arrangement. Such signals according to the invention are in particular, but not exclusively, ratiometric signals as well as lifetimes and decay times.

In some configurations of the invention, each sensor is at least as large as or larger than the detection region of the at least one measuring arrangement which detects the at least one signal from the at least one sensory component contained in the sensor.

In an advantageous configuration of the invention, the detection time required by a measuring arrangement to detect at least one signal of a sensory component is shorter than the dwell time of the sensor containing the sensory component in the detection region of the measuring arrangement.

In some configurations of the invention, the detection time required by a measuring arrangement to detect at least one signal of a sensory component is shorter than the dwell time of the segment of a sensor containing the sensory component in the detection region of the measuring arrangement. Advantageously, this applies in particular to sensors that contain a plurality of sensory components arranged in segments.

In an advantageous configuration of the invention, for at least one detection time of the measuring arrangement detecting a particular sensory component, there are repeatedly no sensors or sensor segments containing that very sensory component in the detection region of that very measuring arrangement detecting the particular sensory component.

In an advantageous configuration of the invention, one sensor according to the invention or a plurality of sensors according to the invention are passively moved by the movement of the reactor content or reaction mixture occurring in the course of the mixing of the reactor and thereby get into the detection region of at least one measuring arrangement and also get out of the detection region of at least one measuring arrangement. Such methods according to the invention result in particular from the use of sensors according to the invention in shaken reactors (for example shake flasks, bags, culture plates, microtiter plates, T-flasks), in stirred tank reactors and in bubble column reactors.

In some embodiments of the invention, at least one sensor according to the invention contains at least one marker that can be detected by at least one measuring arrangement via at least one marker signal. In an advantageous configuration of such embodiments, the marker triggers via its marker signal the start of the detection of at least one signal of at least one sensory component by at least one measuring arrangement. In an advantageous configuration of embodiments of the invention having at least one marker, sensors that are used to monitor different properties are also equipped with markers that have different marker signals, in particular but not exclusively, to trigger different detection methods of the measuring arrangement or to be able to uniquely assign the different sensors to the respective property of the reactor content monitored by them. In some configurations of the invention, at least one property of at least one sensory component detectable by suitable measuring arrangements is used in a sensor as a marker. For example, the luminescence intensity of a luminophore whose luminescence lifetime is detected as a signal can be detected as a marker signal so that a suitable measuring arrangement detects the luminescence lifetime whenever a luminescence marker signal is present.

In an advantageous configuration of the invention, a measuring arrangement can detect different signals from different sensory components and/or different marker signals from different markers. This is carried out in particular by combining different suitable signal detectors and, where necessary, signal exciters.

In some embodiments of the invention, at least one measuring arrangement is attached stationarily with respect to the reactor so that there is no relative movement between the reactor and the measuring arrangement. In other embodiments of the invention, relative movement between the reactor and the at least one measuring arrangement takes place.

In some embodiments of the invention, at least one sensor according to the invention is added into the reactor when filling the reactor. In some embodiments of the invention, the addition of at least one sensor according to the invention to the reactor takes place prior to the start of the process to be carried out in the reactor, for example, prior to the start of the cultivation or incubation of cells or prior to the start of a chemical reaction. In other embodiments of the invention, the addition of at least one sensor according to the invention into the reactor takes place already before packaging or sterilizing the reactor or during the process already running in the reactor. In embodiments requiring high sterility or purity, the addition is done from a sterile addition system for sensors according to the invention, which are then also sterile.

In some embodiments, the sensors of the invention are stored in a storage medium prior to adding them into the reactor, which ensures the shelf life of the sensors and keeps the sensors ready for use.

According to the invention, the shape of each sensor can be adapted to the shape and type of mixing of a reactor or to its reactor content. For example, in shake flasks or other orbitally shaken reactors, sensors in the form of annular segments or shapes derived therefrom and matching the radii of the reactors used or orbital movements can enable optimal movement of the sensors within the reactor. In turbulently stirred stirred tank reactors, on the other hand, spherical or ellipsoidal sensors can be advantageously used.

In some embodiments of the invention, the sensors according to the invention have shaped elements that promote optimal alignment or movement of the sensors in the reactor content or reaction mixture. Such shaped elements are in particular, but not exclusively, wing-, fin- or fin-like structures.

In some embodiments of the invention, at least one sensory component or at least one converting component or at least one sensor matrix comprising at least one sensory component or at least one converting component is set back or inward relative to the outer contour of the sensor such that in the event of a collision or other contact between the relevant sensor and the reactor (in particular the reactor wall or reactor internals), there is no direct contact between the reactor and the set back or inward at least one sensory component, converting component or sensor matrix. Advantageously, this reduces the mechanical load on the at least one sensory component, converting component or sensor matrix that is set back or inwards.

In some embodiments of the invention, a sensor has a geometry which specifically hinders or prevents certain forms of movement in the reactor. In an advantageous embodiment of the invention for shaken reactors, in particular shake flasks, this is achieved by corners or edges in the geometry of the sensor, so that rolling movements correspondingly are impeded and a designed sensor moves preferentially with the reaction mixture under a wide variety of mixing conditions (in particular with regard to shaking conditions, filling level of the reactor and viscosity of the reaction mixture) instead of rolling or sliding over the reactor bottom. In some such embodiments of the invention, the corners and edges are arranged and combined with suitable transitions in such a way that an overall advantageous flow condition is obtained, which also prevents undesired rolling or sliding movements, stabilizes the position of the sensor in the reaction mixture and supports the movement of the sensor with the reaction mixture.

According to the invention, the density of sensors according to the invention can be adapted according to the monitoring objective to the density of the reactor content or reaction mixture in order to monitor properties of the reactor content or reaction mixture in specific regions, for example at the bottom or at the surface of the reaction mixture.

According to the invention, a plurality of sensors of the same type and nature can be used in one reactor. This can be done in particular, but not exclusively, in large reactors in order to shorten the time between the detections of at least one signal of one or more specific sensory components in that corresponding sensors with these very sensory components enter the detection region of suitable measuring arrangements more frequently, thus enabling a closer-meshed monitoring of at least one property of the reactor content.

Sensors according to the invention can comprise one or more converting components which do not emit a signal detectable by measuring arrangements but interact with at least one sensory component to influence in this manner at least one property detectable by a measuring arrangement and thus at least one signal of the sensory component.

In some embodiments of the invention, different sensors according to the invention, each having different converting components, contain the same sensory component as well as different markers to reduce the number or complexity of measuring arrangements required.

In some embodiments of the invention, a plurality of different signals of one or more sensory components or one or more combinations of at least one sensory component and at least one converting component are used to monitor or determine a property of the reactor content.

In some embodiments of the invention, sensors according to the invention are used that are divided into segments, wherein different segments each contain different combinations of at least one marker or at least one sensory component and none, one, or more converting components. In an advantageous configuration of such embodiments, the segments are in a defined order and a defined spacing so that only one marker is required to trigger the sequential detection of the signals of the sensory components from the respective segments of the sensor. In an advantageous configuration of such embodiments, each segment is larger than the detection region of the sensor. In an advantageous configuration of such embodiments, a region without a sensory component is located between adjacent segments. In some embodiments of the invention, at least one marker is located between adjacent segments or on at least one outer surface of a sensor according to the invention.

In some embodiments of the invention, the movement of sensors according to the invention in the reactor content or reaction mixture takes place periodically or approximately periodically so that signals of the respective sensory components can be detected at regular intervals by suitable measuring arrangements. In such embodiments, advantageously, the detection of signals and marker signals can be synchronized with the movement periodicity, in particular but not exclusively, based on previously detected signals and periods calculated therefrom or based on the detection of periodic movement signals, for example accelerations, rotation rates or positions or distribution or movement behavior of the reactor contents or reaction mixture.

In some embodiments of the invention, the detection of at least one signal of at least one sensory component or at least one marker signal is periodic. In some embodiments of the invention, in particular but not exclusively in the case of periodic mixing movements, the detection can advantageously take place at a frequency which is higher than the mixing frequency. Examples of mixing frequencies are agitator speeds, shaking or rocking frequencies and volume flows.

In some other embodiments of the invention, the acquisition of at least one signal of at least one sensory component or at least one marker signal is non-periodic, in particular partially or completely randomized, in order to minimize or eliminate beating, oscillation, aliasing and other acquisition time-dependent phenomena of at least one acquired signal of at least one sensory component or at least one marker signal.

In some embodiments of the invention, the reactor has devices that influence the movement of sensors according to the invention, in particular to influence, increase, or decrease the probability of residence of sensors according to the invention in specific regions of the reactor or reactor content, respectively. In such embodiments, in particular but not exclusively, flow elements, baffles, flaps, fins, grooves, troughs, nets, or targeted fluid flows are used to influence the movement of sensors according to the invention.

In some embodiments of the invention, a plurality of sensory component signals and/or marker signals from one or more sensors are detected simultaneously or temporally overlapping by a plurality of different measuring arrangements, or else by a plurality of different signal detectors or signal exciter-signal detector pairs on the same measuring arrangement.

In some embodiments of the invention, a plurality of detected signals are computed together by suitable algorithms and software on computers to form new signals or correlates, which are then used to monitor at least one property of the reactor content or reaction mixture.

In some embodiments of the invention, different converting components or sensory components or combinations of both are intermixed or otherwise embedded in spatial proximity to each other in a common segment of a sensor. In some embodiments of the invention, such mixtures of different converting components or sensory components or combinations of both are detected together by a suitable measuring arrangement, for example in the case of self-referenced sensors as part of dual lifetime referencing or to compensate for ambient temperature or other environmental properties and parameters.

In an advantageous embodiment of the invention, the measuring arrangement is located outside the reactor and penetrates with its detection region the reactor wall and a portion of the reactor content. In some other embodiments of the invention, the measuring arrangement is designed as an immersion probe and is thus located inside the reactor. In further embodiments of the invention, the measuring arrangement is located completely within the reactor and moves with or against the reactor contents.

According to the invention, influencing the at least one sensory component and its signal detectable by at least one measuring arrangement can be carried out directly or indirectly by the at least one property to be monitored, in particular also by interaction chains with converting components or with other properties or constituents of the reactor content.

In some embodiments of the invention, the same property of the reactor content or reaction mixture to be monitored is redundantly detected by a plurality of different combinations of sensory components and, where necessary, converting components and, where appropriate, different measuring arrangements. Advantageously, this redundancy increases the robustness and quality of the monitoring of the property to be monitored.

In some embodiments of the invention, at least one sensor according to the invention has a layered structure. In some embodiments of the invention, at least one sensor according to the invention has a core-shell structure, wherein a core comprising at least one sensor matrix acts as a carrier of at least one active shell, wherein the active shell comprises at least one sensing component or at least one converting component or at least one marker or combinations of the aforementioned. In some embodiments of the invention, such a core may also comprise at least one sensory component or at least one converting component or at least one marker or combinations of the foregoing, or may itself be layered.

In some embodiments of the invention, at least one sensor according to the invention is constructed in such a way that, during the dwell time of the sensor in the detection range of at least one measuring arrangement, at least one sensory component or at least one converting component or at least one marker or a combination of the aforementioned can be detected or excited or excited and detected by the measuring arrangement, largely or completely independently of its orientation. In some embodiments of the invention, the sensor is therefore rotationally symmetrical.

In some embodiments of the invention, at least one sensor matrix or at least one of its components or at least one component embedded by it is pre-arranged such that background signals from the environment, in particular but not exclusively ambient light, electromagnetic fields or other radiation, are shielded. This reduces the interaction of such background signals with sensory or converting components or markers or with the detection of signals from sensory components or marker signals in the detection range of at least one measuring arrangement during the dwell time of the relevant sensor in the detection range of the at least one measuring arrangement. Examples of such embodiments may include, in particular but not exclusively, optically impermeable or selectively permeable layers or dyes, scattering or absorbing components, and materials with suitable permeability or permittivity.

In some embodiments of the invention, at least one sensor matrix is prepared in such a way that it amplifies at least one signal of at least one sensory component or at least one marker signal or at least one signal excitation, in particular but not exclusively by means of scattering or reflective layers or scattering or reflective particles incorporated in at least one sensor matrix.

The present invention is explained in more detail with reference to the figures and exemplary embodiments.

### Exemplary Embodiments

Identical or identically acting elements in the figures are designated by identical reference signs, with only those reference signs being used in each case that are necessary for understanding the figure, also in the context of the other figures. Duplicate designations in the same or similar constituents of a figure are therefore largely omitted.

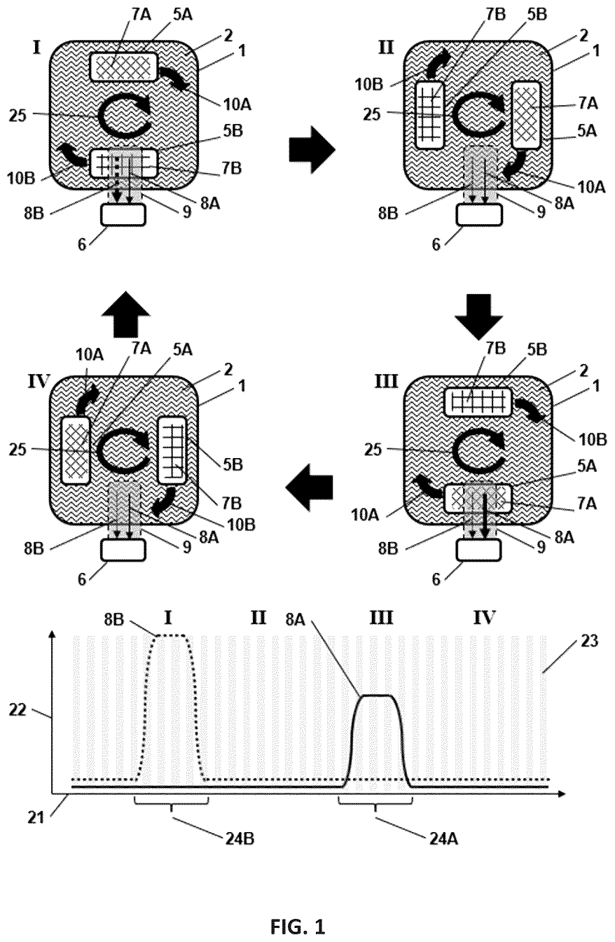

FIG. 1 shows a schematic illustration of the method according to the invention using two sensors 5 with different sensory components 7. The method according to the invention is carried out in a reactor 1, the reactor content 2 of which is mixed as a result of at least one mixing movement 25. Two sensors 5A and 5B are located in the reactor, each containing a sensory component 7A and 7B. According to the invention, the two sensors 5A and 5B are used to monitor two properties of the reactor content 2, wherein in each case one property of the reactor content 2 influences in each case one sensory component 7A and 7B, respectively, in such a manner that a signal 8A and 8B, respectively, emanating from the respective sensory component 7A and 7B, respectively, can be detected by the measuring arrangement 6. In this context, the executed method according to the invention is characterized in that the sensors 5A and 5B used are not stationary and carry out a movement 10A and 10B, respectively, within the reactor 1 as a result of the mixing movement 25 so that the sensors 5A and 5B used are not permanently located in the detection region 9 of the measuring arrangement 6, but from time to time, in the case of periodic movements 10 also periodically, remain in the detection region 9 of the measuring arrangement 6 for a certain dwell time 24 and can be detected by the latter with regard to the signals 8 of their sensory components 7.

In the embodiment of the method according to the invention shown in FIG. 1, signals 8 from the detection region 9 are detected by the measuring arrangement 6 at regular intervals over a certain detection time 23. The embodiment of the method according to the invention shown in FIG. 1 uses detection times 23 that are shorter than the dwell time 24 of the respective sensor 5A and 5B, respectively. As a result and in combination with a periodically repeated detection of signals 8 by the measuring arrangement 6 it is ensured, even without the use of a marker 13, that according to the invention, the detection of the respective signals 8A and 8B, respectively, of the two sensory components 7A and 7B, respectively, required for monitoring the reactor content 2 is carried out by the measuring arrangement 6 while the respective sensor 5A and 5B, respectively, is located in the detection region 9 of the measuring arrangement 6. The measuring arrangement 6 shown is designed in such a manner that it can detect different signals 8, but in particular the signals 8A and 8B, respectively, of the two sensory components 7A and 7B, respectively.

FIG. 1 shows in the upper and middle region four snapshots I, II, III and IV from a continuous mixing movement 25 of the reactor content 2 in the reactor 1, which in its course over time 21 causes the non-stationary sensors 5A and 5B to perform a movement 10 within the reactor 1. In the lower region of FIG. 1, the course of the intensities 22 of the signals 8A and 8B, respectively, of the two sensory components 7A and 7B, respectively, detected by the measuring arrangement 6 over time 21 is shown schematically, the times of the four snapshots being marked with the respective Roman numerals I, II, III and IV.

The method according to the invention can be seen in FIG. 1. In FIG. 1, sensor 5A is not located in the detection region 9 of the measuring arrangement 6 so that the intensity 22 of the signal 8A is so low that it is not used for determining the property of the reactor content 2 to be monitored by sensor 5A with the sensory component 7A. The same applies to the snapshot in FIG. 1, III in which sensor 5B is not located in the detection region 9 of the measuring arrangement 6 so that the intensity 22 of the signal 8B is so low that it is not used for determining the property of the reactor content 2 to be monitored by sensor 5B with the sensory component 7B. In the snapshots shown in FIG. 1, II and IV, no sensor 5 is located in the detection region 9 of the measuring arrangement 6 so that both signals 8A and 8B are so low that they are not used for determining the property of the reactor content 2 to be monitored by sensors 5A and 5B with their sensory components 7A and 7B. In FIG. 1, I, the sensor 5B is located in the detection region 9 of the measuring arrangement 6 so that the intensity 22 of the signal 8B is so high that it is used for determining the property of the reactor content 2 to be monitored by sensor 5B with its sensory component 7B. As a result of the short detection time 23 compared to the dwell time 24B of the sensor 5B in the detection region 9 of the measuring arrangement 6, the signal 8B suitable for monitoring can be reliably and robustly identified. The same applies to the situation of the sensor 5A in the detection region 9 of the measuring arrangement 6 shown in FIG. 1, III, which here too produces a signal 8A suitable for monitoring the property of the reactor contents 2 to be monitored. Thus, in the illustrated embodiment, the respective intensity 22 of the signal 8A and 8B, respectively, is used as a marker signal 14 for the respective sensor 5A and 5B, respectively.

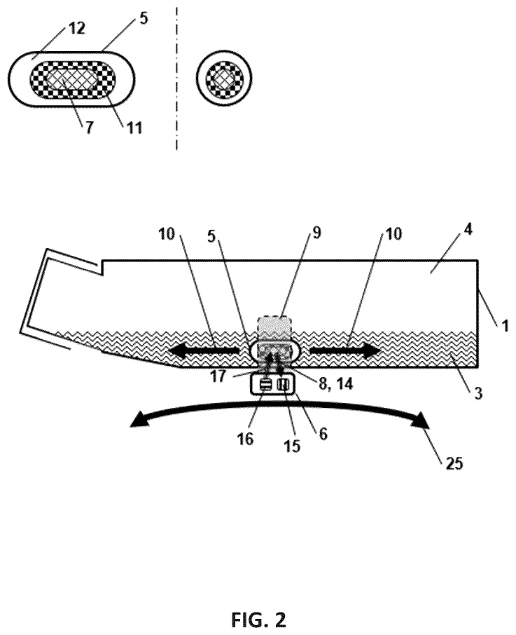

FIG. 2 shows a schematic illustration of a sensor 5 according to the invention and a device according to the invention for carrying out the method according to the invention using the example of a T-flask as reactor 1 for cultivating cells. Shown as a side section (top left) and cross section (top right) is a capsule-shaped sensor 5 containing a sensory component 7 encased by a converting component 11. The sensory component 7 and the converting component 11 are embedded in a sensor matrix 12. According to the invention, the sensory component 7 generates at least one signal 8 in the detection region 9 of a suitable measuring arrangement 6, which signal 8 is dependent on at least one property of the reactor content 2. Typical examples of such properties are known to those skilled in the art and include, in particular but not exclusively, oxygen concentration, pH, polarity of the environment, salinity, temperature, pressure and many more. Sensory components 7 according to the invention which can generate signals 8 according to the invention in dependence on these very properties are in particular, but not exclusively, dyes and luminophores which generate optical signals 8, but also metallic or semiconductor-derived nano- or microparticulate structures, bioconjugates or systems combined with gels, which can generate capacitive signals 8, more complex impedance signals 8 which can be detected in particular in frequency-dependent manner, or magnetic signals 8.

By the embodiment of the invention shown in FIG. 2 with encasement of the sensory component 7 by a converting component 11, any properties of the reactor content 2 can be monitored according to the invention by using at least one suitable converting component 11 in combination with at least one sensory component 7. According to the invention, the at least one property of the reactor content 2 to be monitored interacts here with at least one converting component 11 and thereby influences at least one property of the sensory component 7 that can be detected by a measuring arrangement 6. Typical examples of such converting components 11 are in particular, but not exclusively, enzymes, antibodies, ribozymes, catalysts, nucleic acids and derivatives thereof, as well as aptamers, wherein the interaction between the property to be monitored (for example, a substance concentration) with at least one converting component 11 occurs in particular by specific association and dissociation processes or specific reactions, which are often catalytically influenced. Such interactions according to the invention between at least one property of the reactor content 2 to be monitored and at least one converting component 11 influence locally, in particular in the immediate vicinity of the converting component 11, at least one property of the reactor content 2, which in turn influences at least one signal 8 of at least one sensory component 7 located in the interaction region of the converting component 11.

The embodiment of the sensor 5 shown in FIG. 2 with a converting component 11 encasing the sensory component 7 advantageously enhances the influence of the converting component 11 on the sensory component 7. For the embodiment shown in FIG. 2 as well as for similar or derived embodiments of the invention, oxygen- or pH-dependent luminophores or luminophore systems are used in particular, but not exclusively, as sensory component 7 in combination with oxygen- or proton-consuming or -generating enzymes and enzyme reactions as converting component 11. Specific binding antibodies or aptamers can also be used as converting component 11 together with metallic or semiconducting particles or particle systems as sensory component 7, in particular if the latter have a suitable surface plasmon resonance for optical detection or suitable magnetic, capacitive or resistive properties for, in particular, frequency-dependent impedance detection.

The sensor matrix 12 shown in FIG. 2 not only serves for embedding the converting component 11 and the sensing component 7, but also provides mechanical stability to the sensor 5 according to the invention. Furthermore, the sensor matrix 12 can also be designed as a partially or fully substance-selective structure to increase the specificity or sensitivity of the at least one converting component 11 or the at least one sensory component 7. Such embodiments of the sensing matrix 12 comprise in particular, but not exclusively, hydrogels and membranes with defined pore size and distribution or with defined polarity, hydrophobicity, oxygen or proton permeability.

FIG. 2 illustrates the method according to the invention using the example of a T-flask as reactor 1, the reactor content 2 of which is formed by a cell culture suspension as reaction mixture 3 and by a gas-filled headspace 4 lying thereabove. A sensor 5 is located in the reaction mixture 3 in the embodiment shown in FIG. 2 above. Outside the reactor 1 is a measuring arrangement 6 which comprises at least one signal exciter 16 and at least one signal detector 15. At least one combination of at least one signal exciter 16 and at least one signal detector 15 spans at least one detection region 9 of the measuring arrangement 6, which penetrates through the wall of the reactor 1 into the reactor content 2 so that the signal excitation 17 of at least one sensor 5 according to the invention can take place there via the at least one sensory component 7 contained therein, and the signal 8 thereof or else a marker signal 14 can be detected by at least one signal detector 15 of the measuring arrangement 6. The T-flask as reactor 1 shown in FIG. 2 is subjected to a mixing movement 25 according to the invention by shaking or rocking, which also effects a movement 10 of the sensor 5 via the resulting movement of the reaction mixture 3 and the reactor 1, so that the sensor moves according to the invention into and also out of the detection region 9 of the measuring arrangement 6.

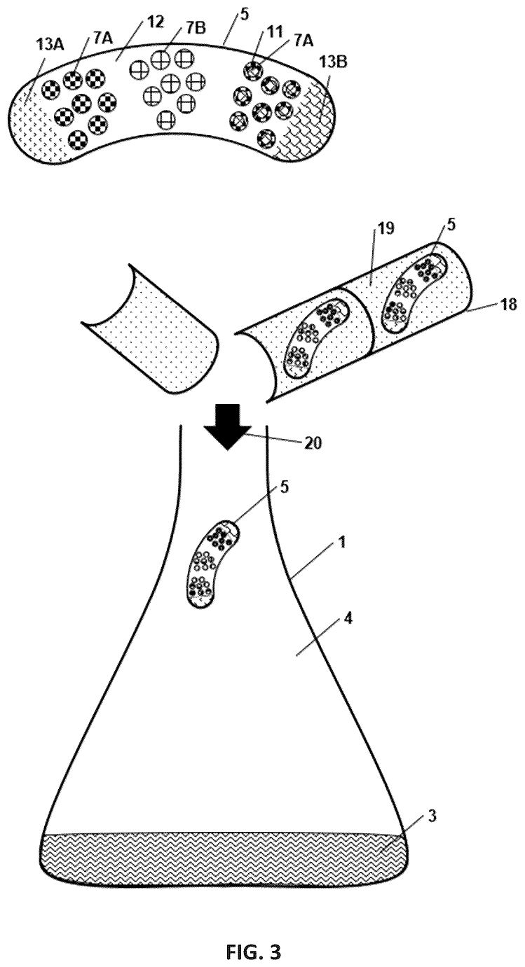

FIG. 3 shows a schematic illustration of a sensor 5 according to the invention for carrying out the method according to the invention using the example of a shake flask as reactor 1 for cultivating cells with an addition system 18 for sensors 5. The sensor 5 shown in FIG. 3 is arc-shaped in order to be able to carry out a uniform movement 10 along the round geometry of the wall of the shake flask as reactor 1 with as little collision as possible. Markers 13A and 13B are embedded in the sensor matrix 12 in the front and rear areas of the sensor 5. Between the regions with markers 13A and 13B are several regions with different sensory components 7A and 7B as well as a converting component 11, wherein the components 7A and 7B or, respectively, their combinations (7A encased by 11) are configured as particles embedded in the sensor matrix 12. In each region of the sensor 5 shown in FIG. 3 there is at most one type of particle. One region of the illustrated sensor 5 contains particles of the sensory component 7A which generates, for example, an oxygen-dependent signal 8A. One region of the illustrated sensor 5 contains particles of the sensory component 7B which generates, for example, a pH-dependent signal 8B. One region of the illustrated sensor 5 contains combined particles from the sensory component 7A, each of which is encased by a converting component 11 and generates, for example, an oxygen-dependent signal 8A, wherein the local oxygen concentration in the region of influence of the encased sensory component 7A is influenced by an oxidoreductase (e.g. for glucose, lactate, glutamine) as the encasing converting component 11. The individual regions of the sensor 5 are segmented so that the different signals 8 can be assigned locally in the course of the detection, or even afterwards, and thus a distinction can be made between, for example, the oxygen concentration and the glucose concentration in the reactor content 2 determined under local oxygen consumption. As a result of the use of different markers 13A and 13B with different marker signals 14A and 14B in the front and rear regions of the sensor 5, the regions of the sensor 5 containing sensory components 7 can be distinguished regardless of the orientation of the sensor 5.

The lower section of FIG. 3 shows a schematic illustration of a shake flask as reactor 1 before the start of the mixing according to the invention as a result of a mixing movement 25. The reactor 1 comprises a reactor content 2 which is formed by a cell culture suspension as reaction mixture 3 and by a gas-filled headspace 4 lying thereabove. Above the reactor 1, an addition system 18 for sensors 5 is shown, which is divided into individual segments, each segment containing at least one sensor 5 and a storage medium 19. In some configurations of the invention, the sensors 5 and the storage medium 19 are sterile. In the embodiment of the invention shown in FIG. 3, the addition 20 of at least one sensor 5 into the reactor 1 is carried out as integral part of the method according to the invention. Here, the advantageous simplification of the handling of sensors 5 in contrast to the prior art also becomes apparent since the sensors do not have to be attached to the reactor wall. In particular, in embodiments of the invention in which sterile sensors 5 are to be used under sterile conditions in reactor 1, the use of addition systems 18 for sensors 5 according to the invention significantly simplifies the handling of sensors 5.

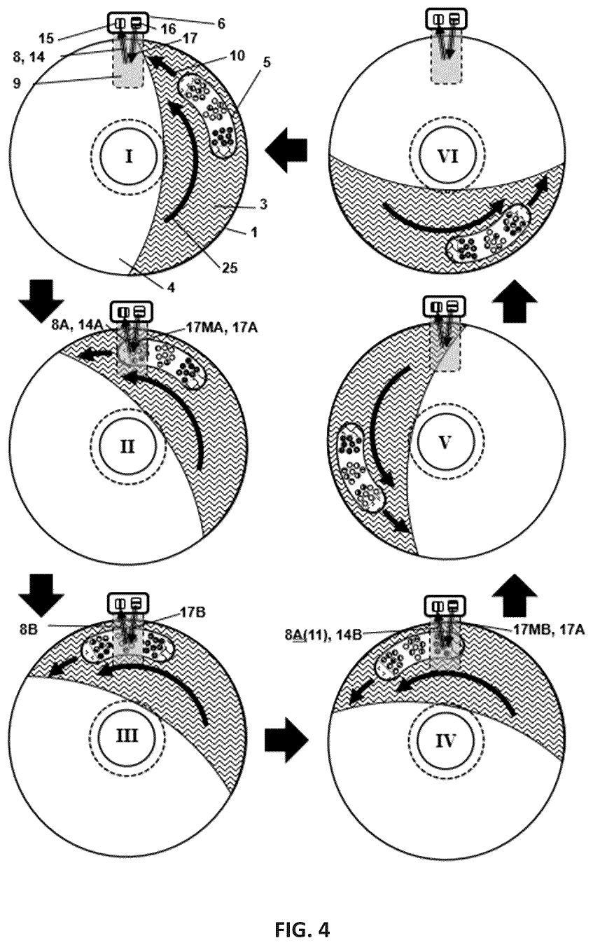

FIG. 4 shows a schematic illustration for carrying out the method according to the invention on a shake flask as reactor 1 for cultivating cells using the sensor 5 according to the invention shown in FIG. 3. Shown are six snapshots (Roman numerals I to VI) of a schematic top view of a shake flask with lid (dashed circle) as reactor 1, the reactor content 2 of which is formed by a cell culture suspension as reaction mixture 3 as well as by a gas-filled headspace 4 lying thereabove and also next to it in the shaking process. In the reaction mixture 3 there is a sensor 5 in the embodiment shown at the top in FIG. 3, so that for the sake of clarity a numerical designation of the sensor components is dispensed with and reference is made to FIG. 3. Outside the reactor 1 is a measuring arrangement 6 which comprises at least one signal exciter 16 and at least one signal detector 15. In the embodiment shown, the measuring arrangement 6 comprises a plurality of signal exciters 16 and signal detectors 15 operating in parallel so that the various markers 13A and 13B as well as the sensory components 7A and 7B and the sensory component 7A encased with a converting component 11 can be detected by a single measuring arrangement 6. For the sake of clarity, only one signal exciter 16 and one signal detector 15 are shown in FIG. 3 for simplicity. At least one combination of at least one signal exciter 16 and at least one signal detector 15 spans at least one detection region 9 of the measuring arrangement 6, which penetrates through the wall of the reactor 1 into the reactor content 2 so that the signal excitation 17 of at least one sensor 5 according to the invention can take place there via the at least one sensory component 7 contained therein, and its signal 8 or else a marker signal 14 can be detected by at least one signal detector 15 of the measuring arrangement 6.

The shaking flask shown in FIG. 4 as reactor 1 is subjected to a mixing movement 25 according to the invention by orbital shaking which, via the resulting periodic rotational movement of the reaction mixture 3, also causes a movement 10 of the sensor 5 so that according to the invention, the latter moves into and out of the detection region 9 of the measuring arrangement 6. In FIG. 4, I, the reaction mixture 3 moves straight into the detection region 9 of the measuring arrangement 6. Since there is no sensor 5 in the detection region 9, neither a valid marker signal 14 nor a valid signal 8 of a sensory component 7 is detected by the measuring arrangement 6. If the reaction mixture 3 continues to move, the sensor 5 enters the detection region 9 of the measuring arrangement 6. First, as shown in FIG. 4, II, the region of the sensor 5 with marker 13A enters the detection region 9 so that the orientation of the sensor 5 can be determined based on the marker signal 14A detected by the measuring arrangement 6 by means of the signal excitation 17MA. In addition, the signal exciters 16 and signal detectors 15 for the following sensory components 7A and 7B are now activated so that at the moment of the presence of the respective sensory component 7 in the detection region 9 of the measuring arrangement 6, a respective valid signal 8 can be detected in order to monitor through this signal the property of the reaction mixture 3 associated therewith according to the invention. Since the various particles with sensory components 7 are arranged one behind the other in a segmented manner in the depicted sensor 5 and move through the detection region 9 of the measuring arrangement 6 one after the other as a result of the movement 10 of the sensor 5, the measuring arrangement 6 detects the signals 8 of the sensory components 7 one after the other, first, using signal excitation 17A, the signal 8A of the sensory component 7A (FIG. 4, II), then, using signal excitation 17B, the signal 8B of the sensory component 7B (FIG. 4, III), then, using signal excitation 17A, the signal 8A of the sensory component 7A encased with converting component 11 (FIG. 4, IV). Finally, the marker signal 14B of the marker 13B is detected by means of the signal excitation 17MB (FIG. 4, IV), whereby a validation of the positioning of the sensor 5 during its movement 10 through the detection region 9 is also carried out once again. Subsequently, the sensor 5 moves out of the detection region 9 of the measuring arrangement 6 (FIG. 4, V-VI). As a result of the periodic mixing movement 25 of the shaking process illustrated here, the movement 10 of the sensor 5 is also subject to a certain periodicity. Based on the detection of the mixing movement 25 or the movement 10 of the sensor 5, the detection of the individual signals 8 and marker signals 14 can be synchronized in some embodiments of the invention. In addition, the use of several different markers 13 with correspondingly different marker signals 14 in different segments of a sensor 5 allows an unambiguous assignment of different signals 8 to their respective properties of the reactor content 2 or reaction mixture 3, respectively, to be monitored, even if the position and orientation of the sensor 5 in the detection region 9 changes as a result of the movement 10 of a sensor 5.

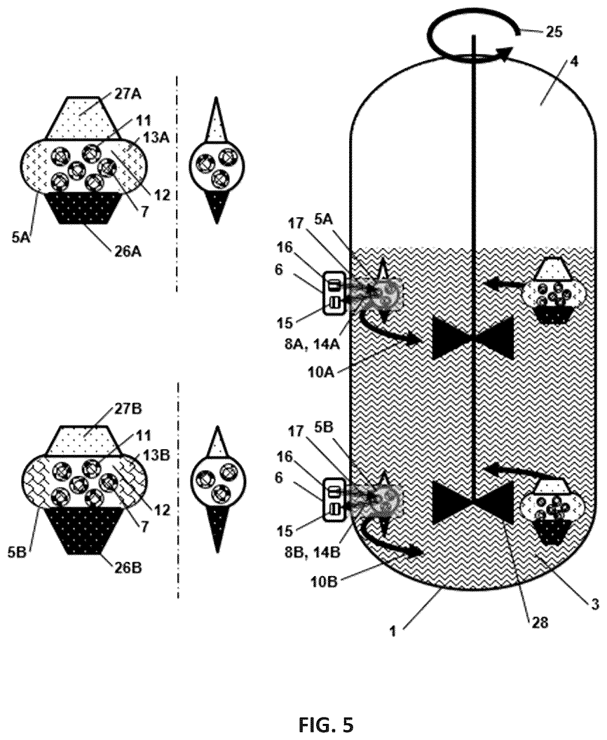

FIG. 5 shows a schematic illustration of two sensors 5 according to the invention and a device according to the invention for carrying out the method according to the invention on a stirred tank reactor as reactor 1. The sensors 5A and 5B shown contain sensory components 7 configured as particles which are surrounded by a converting component 11 and embedded in the sensor matrix 12. Sensor 5A further contains markers 13A in two regions, the marker signal 14A of which is different from the marker signal 14B of the marker 13B contained in sensor 5B so that sensors 5A and 5B can be distinguished based on their marker signals 14A and 14B. Furthermore, the sensors 5A and 5B each comprise a fin 26 with a density that is higher than that of the reaction mixture 3 and a fin 27 with a density that is lower than that of the reaction mixture 3. According to the invention, these fins 26 and 27 are used in particular for flow-dynamic stabilization and positioning of the sensors 5. Furthermore, by suitable dimensioning of the fins 26 and 27, the average density of a sensor 5 can be determined and thus the probability of presence of the sensor 5 in certain regions of the reactor content 2 or the reaction mixture 3, respectively, can be systematically influenced.

The sensor 5A shown at the top left in FIG. 5 is equipped with a large fin 27A of low density and a small fin 26A of high density so that the average density of the sensor 5A is lower than the density of the reaction mixture 3 of the reactor 1 shown on the right in FIG. 5. Therefore, sensor 5A stays predominantly in the upper part of the reaction mixture 3, even during turbulent mixing, and can thus be used systematically for monitoring properties of this upper region of the reaction mixture 3. Sensor 5B, shown at the bottom left of FIG. 5, is equipped with a small fin 27B of low density and a large fin 26B of high density so that the average density of sensor 5B is higher than the density of reaction mixture 3 of reactor 1 shown on the right of FIG. 5. Therefore, sensor 5B stays predominantly in the lower part of reaction mixture 3, even during turbulent mixing, and can thus be used systematically to monitor properties of this lower region of the reaction mixture 3. The use of fins 26 and 27 of different densities, with the fin 26 of higher density preferably being oriented facing downwards and the fin 27 of lower density preferably being oriented facing upwards, stabilizes the position of the respective sensor 5 in the reaction mixture 3 and thus also in the detection region 9 of suitable measuring arrangements 6, whereby in some embodiments of the invention a better quality of signals 8 and marker signals 14 is achieved.

FIG. 5 schematically shows on the right an embodiment of the invention using a stirred tank reactor as reactor 1 with the sensors 5A and 5B shown on the left in FIG. 5. Shown is a stirred tank reactor as reactor 1, the reactor content 2 of which is formed by a cell culture suspension as reaction mixture 3 and a gas-filled headspace 4 lying thereabove. Two sensors 5A and 5B are located in the reaction mixture 3 in the embodiment shown on the left in FIG. 5, with the sensors 5A being located predominantly in the upper region and the sensors 5B being predominantly located in the lower region of the reaction mixture 3. Outside the reactor 1, there is in each case one measuring arrangement 6 for both the upper and lower regions of the reaction mixture 3, each of which comprises at least one signal exciter 16 and at least one signal detector 15. In the embodiment shown, each measuring arrangement 6 comprises a plurality of signal exciters 16 and signal detectors 15 operating in parallel so that the various markers 13A and 13B as well as the sensory component 7A encased with a converting component 11 can be detected by a single measuring arrangement 6. For the sake of clarity, only one signal exciter 16 and signal detector 15 per measuring arrangement 6 is shown for simplicity. At least one combination of at least one signal exciter 16 and at least one signal detector 15 spans at least one detection region 9 of the respective measuring arrangement 6, which penetrates through the wall of the reactor 1 into the reaction mixture 3 so that the signal excitation 17 of at least one sensor 5 according to the invention can take place there via the at least one sensory component 7 contained therein, and its signal 8 or else a marker signal 14 can be detected by at least one signal detector 15 of the measuring arrangement 6. The reactor 1 shown has an agitator 28 and is subjected by the latter to a mixing movement 25 according to the invention which, via the resulting movement of the reaction mixture 3, also causes a movement 10 of the sensors 5A and 5B so that according to the invention, they move into and also out of the detection region 9 of the respective measuring arrangement 6. By assigning the measuring arrangements 6 to the respective upper and lower regions of the reaction mixture 3, according to the invention, the properties of the latter can now be monitored with spatial resolution by means of the sensors 5A and 5B via their signals 8A and 8B, wherein the use of different markers 13A and 13B, via their different marker signals 14A and 14B, allows the two sensors 5A and 5B to be distinguished if they should ever leave their target residence region in the reaction mixture 3.

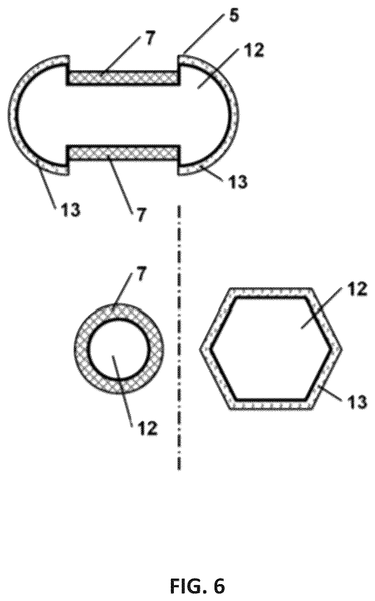

FIG. 6 shows a schematic representation of a sensor 5 according to the invention in a core-shell design with adapted geometry for use in orbitally shaken systems. The sensor matrix 12 forms the mechanically stable core of the sensor 5 and acts as a carrier for the marker 13 and the sensory component 7. FIG. 6 shows a longitudinal section of a sensor 5 according to the invention at the top and two cross-sections at the bottom, on the left a cross-section through the central area of the sensor 5 and on the right a cross-section through one of the edge caps of the sensor 5. In the version shown, the sensory component 7 is set back inwards in such a way that even when the sensor 5 comes into contact with the reactor 1, it does not touch the latter and is therefore largely protected from mechanical damage. In contrast, the two end caps coated with marker 13 clearly stand out and thus form the main points of contact between the sensor 5 and the reactor 1.

In order to prevent the sensor 5 from rolling over the bottom of the reactor 1, particularly during shaking operation, and instead to move the sensor 5 through the reactor 1 in a controlled manner due to the movement of the reaction mixture 3, the two end caps of the sensor 5 are angular, hexagonal in the embodiment shown in FIG. 6. In order to achieve an optimal and stable position and orientation of the sensor 5 in the moving reaction mixture 3 for detection by at least one suitable measuring arrangement 6, the sensor 5 is adapted to the flow conditions in the reaction mixture 3.

In the embodiment shown in FIG. 6, the sensor 5 is rotationally symmetrical and has a length-to-diameter ratio of more than one, preferably at least two. Furthermore, the embodiment of the sensor 5 according to the invention shown in FIG. 6 has a flowing transition of the hexagonal edge structures of the two end caps carrying the marker 13 into an edge-free geometry, in this case a spherical segment, which is more advantageous in terms of fluid mechanics.

The depth of and the transition into the reset of the sensor component 7 can also be advantageously selected so that in the application there is an optimum compromise between the avoidance of direct contact between the sensor component 7 or other mechanically sensitive components of the sensor 5 and the reactor 1 and an optimum flow to the sensor 5 for the application, in particular but not exclusively through the use of suitable geometries to avoid stalls and vortices behind the end caps of the sensor 5 against which the flow passes. In an advantageous embodiment of the invention, the depth of the recess of the sensory component 7 is also selected to be just small enough to ensure mechanical protection of sensitive components of the sensor 5 by avoiding contact with the reactor 1 and optimizing the flow, while at the same time ensuring that as little reaction mixture 3 as possible is between the sensory component 7 and the at least one suitable measuring arrangement 6 during the detection of at least one signal 8 of at least one sensory component 7. This is important for applications in which, in particular but not exclusively, the turbidity or absorption of the reaction mixture 3 changes over time, for example due to cell growth or the formation or dissolution of substrates, products, by-products, metabolites, foams, particles, emulsions or other phase mixtures. The sensor 5 according to the invention shown in FIG. 6 can, for example, be designed as a sensor for dissolved oxygen, wherein an oxygen-quenchable luminophore as sensory component 7 is applied as a thin layer to a core of polyamide as primary sensor matrix 12, wherein the end caps of this primary sensor matrix 12 also act as carriers of another luminophore as marker 13. The sensory component 7 and the marker 13 can also be embedded in one or more suitable sensor matrices 12 and applied to the core as the primary sensor matrix 12 by means of these.

### REFERENCE LIST

For the respective interpretation of the reference signs, the description and claims are to be observed. Reference signs supplemented with letters designate separate or different elements of the same type.
1 reactor
2 reactor content
3 reaction mixture
4 headspace
5 sensor
6 measuring arrangement
7 sensory component with at least one property detectable by the measuring arrangement 6
8 signal of a sensory component 7 detectable by a measuring arrangement 6
9 detection region of a measuring arrangement 6
10 movement of a sensor 5
11 converting component interacting with at least one sensory component 7 or at least one converting component
12 sensor matrix
13 marker
14 marker signal
15 signal detector
16 signal exciter
17 signal excitation
18 addition system
19 storage medium
20 addition of at least one sensor 5 into a reactor 1
21 time
22 intensity of signals 8 and marker signals 14, respectively
23 detection time
24 dwell time
25 mixing movement
26 fin with higher density compared to the reactor content 2 or reaction mixture 3
27 fin with lower density compared to the reactor content 2 or reaction mixture 3
28 agitator

1. A method for monitoring the content of mixed reactors 1,
wherein the content has at least one property to be monitored,
wherein the at least one property to be monitored directly or indirectly influences at least one signal of at least one sensory component,
wherein the at least one signal of the at least one sensory component is detectable by at least one measuring arrangement,
wherein the at least one sensory component is a constituent of at least one sensor,
the at least one sensor is not stationary and performs a movement in the reactor, and
at least one detection of the at least one signal of the at least one sensory component is carried out by at least one measuring arrangement while the at least one sensor is located in the detection region of the at least one measuring arrangement, and
the at least one sensor is not permanently located in the detection region of the at least one measuring arrangement.

2. The method according to claim 1, wherein the detection time of the at least one signal is shorter than dwell time of the at least one sensor in the detection region of the at least one measuring arrangement.

3. The method according to claim 1, wherein the monitoring is carried out using at least one converting component contained in the sensor, wherein the at least one converting component directly or indirectly influences at least one sensory component contained in the sensor and the signal thereof detectable by at least one measuring arrangement.

4. The method according to claim 1, wherein a plurality of different sensors is used to monitor a plurality of different properties of the reactor contents.

5. The method according to claim 1, wherein at least one marker signal of at least one marker contained in the sensor is detected by at least one measuring arrangement.

6. The method according to claim 1, wherein a measuring arrangement comprising a plurality of signal detectors and/or a plurality of signal exciters detects a plurality of signals and/or a plurality of marker signals.

7. The method according to claim 1, wherein the addition of at least one sensor into the reactor takes place before the start of the process to be carried out in the reactor.

8. A device for carrying out the method according to claim 1, wherein:
the device comprises a reactor with reactor content, and
the device comprises at least one non-stationary sensor, and
the device comprises at least one measuring arrangement for detecting at least one signal of at least one sensory component contained in the sensors, and
the volume of at least one detection region of at least one measuring arrangement, which volume overlaps with the reactor content, is smaller than the volume of the reactor content.

9. The device according to claim 8, wherein the volume of at least one detection region of at least one measuring arrangement, which volume overlaps with the reactor content, is smaller than at least one of the sensors detectable by the measuring arrangement.

10. The device according to claim 8, wherein the device comprises a plurality of non-stationary sensors.
